(19) United States
(12) Patent Application Publication
(10) Pub. No.: US 2009/0270652 A1
(43) Pub. Date: Oct. 29, 2009

(54) PROCESS FOR TRANSFERRING HEAT TO A LIQUID COMPRISING DISSOLVED MONOMERIC ACRYLIC ACID, MICHAEL ACRYLIC ACID OLIGOMERS AND ACRYLIC ACID POLYMER

(75) Inventors: Till BLUM, Kuantan (MY); Peter ZUROWSKI, Landau (DE); Steffen RISSEL, Kirchheim (DE); Sylke HAREMZA, Neckargemuend (DE); Thorsten FRIESE, Mannheim (DE); Ulrich JAGER, Roemerberg (DE); Volker SCHLIEPHAKE, Schifferstadt (DE); Klaus Joachim MUELLER-ENGEL, Stutensee (DE); Bernd RUMPF, Hockenheim (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/429,717

(22) Filed: Apr. 24, 2009

Related U.S. Application Data
(60) Provisional application No. 61/048,334, filed on Apr. 28, 2008.

(30) Foreign Application Priority Data
Apr. 28, 2008 (DE) 10 2008 001 435.4

(51) Int. Cl. C07C 51/43

(52) U.S. Cl. 562/600

(57) ABSTRACT

A process for transferring heat to a liquid F comprising dissolved monomeric acrylic acid, Michael acrylic acid oligomers, and acrylic acid polymer with the aid of an indirect heat exchanger to which are supplied the liquid F with a temperature T^{F} of ≧150° C. and a fluid heat carrier W with a temperature of T^{W}>T^{F}, wherein the formation of gas bubbles and/or of thin layers of liquid F adjoining a gas phase is brought about in the liquid F during the flow through the heat exchanger.

The present invention relates to a process for transferring heat to a liquid F comprising dissolved monomeric acrylic acid, Michael acrylic acid oligomers and acrylic acid polymer with the aid of an indirect heat exchanger having at least one primary space and at least one secondary space separated from the at least one primary space by a material dividing wall D, in which the liquid F flows through the at least one secondary space, while the at least one primary space is simultaneously flowed through by a fluid heat carrier W, where the liquid F flows into the at least one secondary space with a temperature T^{F}≧150° C. and the fluid heat carrier W into the at least one primary space with a temperature T^{W}>T^{F}. The material dividing wall D serves as a surface for transferring heat from the at least one primary space into the at least one secondary space.

Acrylic acid is an important intermediate which finds use, for example, in the preparation of polymer dispersions (if appropriate also in the form of their esters with alkanols) and of water-superabsorbing polymers.

Acrylic acid is obtainable, inter alia, by heterogeneously catalyzed gas phase partial oxidation of C₃ precursor compounds of acrylic acid (this term is intended to encompass, more particularly, those chemical compounds which are obtainable in a formal sense by reduction of acrylic acid; known C₃ precursors of acrylic acid are, for example propane, propene, acrolein, propionaldehyde and propionic acid; however, the term is also intended to comprise precursor compounds of the aforementioned compounds, for example glycerol (proceeding from glycerol, acrylic acid can be obtained for example, by heterogeneous catalyzed oxidative dehydration in the gas phase; cf., for example, EP-A 1 710 227, WO 06/114506 and WO 06/092272)) with molecular oxygen over catalysts present in the solid state at elevated temperature (cf. for example, German application 102007055086.5 and German application 102006062258.8).

Owing to numerous parallel and subsequent reactions which proceed in the course of the catalytic gas phase partial oxidation and owing to the inert diluent gases which also have to be used in the course of the partial oxidation, pure acrylic acid is not obtained in the catalytic gas phase partial oxidation, but rather a reaction gas mixture (a product gas mixture) which comprises essentially acrylic acid, the inert diluent gases and by-products, and from which the acrylic acid has to be removed.

Typically, one way of removing the acrylic acid from the reaction gas mixture is to first convert the acrylic acid from the gas phase to the condensed (liquid) phase by employing absorptive and/or condensative measures. The further removal of the acrylic acid from the liquid phase thus obtained is subsequently typically undertaken by means of extractive, distillative and/or crystallizative processes.

Alternatively, acrylic acid can also be prepared by homogeneously catalyzed processes proceeding from for example, acetylene (e.g. Reppe process) or ethylene (oxycarbonylation). For the removal of the acrylic acid from the resulting reaction mixtures, the above applies in a corresponding manner.

In the aforementioned separation processes, so-called bottoms liquids are generally also obtained, which comprise especially those constituents whose boiling point at standard pressure (1 atm) is above the boiling point of acrylic acid. Such constituents having a higher boiling point than acrylic acid are, for example, phthalic acid, maleic acid, fumaric acid and/or anhydrides of the aforementioned carboxylic acids, which are generally formed as by-products in the course of the gas phase partial oxidation. In addition, these high boilers include polymerization inhibitors such as for example phenothiazine (PTZ), the monomethyl ether of hydroquinone (MEHQ) and, for example, thermal and/or oxidative decomposition products thereof. However, conversion products which are only formed in the course of removal of the acrylic acid also form part of the aforementioned high boilers. These conversion products include especially free-radical polymers of acrylic acid which, in spite of the presence and the additional use of polymerization inhibitors, form in an undesired manner. Such free-radical polymers of acrylic acid shall be encompassed in this document under the term “acrylic acid polymer”. The polymer chains of acrylic acid polymer formed in this way are in many cases also crosslinked with one another.

The constituents of the bottoms liquids having a higher boiling point than acrylic acid also include relatively high molecular weight chemical compounds which form as a result of condensation reactions of different constituents of the product gas mixture of the partial oxidation. These include, in particular the Michael adducts, which form in the liquid phase as a result of reversible Michael addition of acrylic acid to itself and also to the acrylic acid dimers which form (“Michael acrylic acid dimers”, or “Michael diacrylic acid”) or (in general) acrylic acid oligomers (the term “Michael acrylic acid oligomers” in this document always means the corresponding Michael adducts and not the acrylic acid oligomers which form by free-radical polymerization; the latter are encompassed under the term “acrylic acid polymer” already introduced in this document). However, monomeric acrylic acid itself is normally still a constituent of such bottoms liquids, possibly to a not inconsiderable degree.

It is frequently an objective to very substantially recover acrylic acid still present in above-described bottoms liquids, in order thus to increase the acrylic acid yield of the process employed overall for removing acrylic acid from the product gas mixture of the gas phase partial oxidation.

For this purpose, the bottoms liquid is normally brought to an elevated temperature with the aid of an indirect heat exchanger, and recycled with this elevated temperature back into that separating column having separating internals in which it has formed in the course of the thermal separation of another acrylic acid-comprising gaseous and/or liquid mixture and from which it has been withdrawn (the indirect heat exchanger is therefore frequently also referred to as a circulation heat exchanger). Therein, by the route of evaporation of acrylic acid, the monomeric acrylic acid reformed from the Michael acrylic acid oligomers by the thermal action or present in the bottoms liquid from the start is then removed. In general, the withdrawal point and the recycling point will be spaced apart from one another.

A problem in the above-described procedure is that the action of elevated temperature normally, in an undesired manner, has both a promoting action on the undesired free-radical polymerization and a promoting action on the undesired Michael addition of acrylic acid. This applies especially to that time phase during which the bottoms liquid conducted through the indirect heat exchanger is in contact with a heat exchange surface of the indirect heat exchanger having an elevated temperature (this is that material dividing wall of the indirect heat exchanger which separates the primary space and the secondary space of the indirect heat exchanger from one another; while the liquid to be heated flows through the secondary space, a fluid heat carrier having an elevated temperature normally flows simultaneously through the primary space, and releases some of its heat content to the liquid to be heated which flows within the secondary space through the dividing wall).

As a consequence, especially the surface of the material dividing wall which faces the secondary space, said material dividing wall separating the primary space and secondary space from one another, generally undergoes formation of undesired deposition. This inhibits the heat transfer among other properties and reduces the performance of the indirect heat exchanger. At the same time, an increase in the pressure drop is caused. The process therefore has to be interrupted from time to time for the purpose of removing the deposit. However, even if it is possible in this way to restore the performance of the heat exchanger, the deposits formed comprise free-radically polymerized and hence no longer recoverable acrylic acid, which reduces the acrylic acid yield of the process.

In in-house studies, the above-described problems have been found to be particularly serious when the bottoms liquid to be heated by means of the indirect heat exchanger is a liquid F comprising dissolved monomeric acrylic acid, Michael acrylic acid oligomers and acrylic acid polymer, which, on entry into the at least one secondary space of the heat exchanger, has the following contents:
5 to 50% by weight of Michael acrylic acid oligomers,
at least 40% by weight of acrylic acid polymer,
up to 25% by weight of monomeric acrylic acid,
up to 2% by weight of polymerization inhibitor, and
up to 15% by weight of other compounds.

EP-A 854 129 recommends, for the reduction of the above-described fouling, in the case of liquids which comprise acrylic acid, the obligatory use of a forced-circulation flash evaporator for the heating thereof.

In other words, this is an indirect heat exchanger through whose secondary space the liquid to be heated is forcibly conveyed with the aid of a pump such that the liquid completely fills the secondary space, and formation of gas bubbles in the liquid flowing through the secondary space is suppressed by appropriate pressure conditions. The heated liquid leaves the forced-circulation flash evaporator generally through a throttle device to a lower pressure level, at which gas or vapor bubbles can then be formed outside the heat exchanger.

However, the use of forced-circulation flash evaporators is not entirely satisfactory in the case of liquids F. This is the case in particular when the liquid F, proceeding from a temperature T^{F}≧130° C., is to be brought to a higher temperature level with the aid of the forced-circulation flash evaporator. The use of temperatures below 130° C. to remove acrylic acid from liquids F has likewise not been found to be appropriate to the aim from the point of view of reducing fouling. This is the case even when the heat exchange area of the heat exchanger has been increased in a manner appropriate to the aim, in order to cope with reduced temperatures of the fluid heat carrier flowing through the primary space.

In view of the starting situation outlined, it was an object of the present invention to provide an improved process for heating a liquid F, which enables, in the course of heating of the liquid F in the indirect heat exchanger, firstly reduced formation of acrylic acid polymer and, subsequently and simultaneously, increased recovery of monomeric acrylic acid.

Accordingly, a process is provided for transferring heat to a liquid F comprising dissolved monomeric acrylic acid, Michael acrylic acid oligomers and acrylic acid with the aid of an indirect heat exchanger having at least one primary space and at least one secondary space separated from the at least one primary space by a material dividing wall D, in which the liquid F flows through the at least one secondary space, while the at least one primary space is simultaneously flowed through by a fluid heat carrier W, where the liquid F flows into the at least one secondary space with a temperature T^{F}≧130° C. and the fluid heat carrier W into the at least one primary space with a temperature T^{W}>T^{F}, wherein

a) the liquid F on entry into the at least one secondary space comprises
5 to 50% by weight of Michael acrylic acid oligomers,
at least 40% by weight of acrylic acid polymer,
up to 25% by weight of monomeric acrylic acid,
35 up to 2% by weight of polymerization inhibitor, and
up to 15% by weight of other compounds (other than the aforementioned compounds)
and
   b) in the liquid F flowing through the at least one secondary space, during the flow through the at least one secondary space, the formation of gas bubbles and/or of thin layers of the liquid F adjoining a gas phase is brought about.

Unless explicitly stated otherwise in this document, the wording “liquid F” always means the liquid F on entry in the at least one secondary space of the indirect heat exchanger.

The Michael acrylic acid oligomers in the liquid F consist, based on the total amount G of Michael acrylic acid oligomers present in the liquid F, generally predominantly of Michael acrylic acid dimers (in general, their proportion by weight based on the total amount G is from 40 to 60% by weight). Michael acrylic acid trimers account typically for a proportion by weight on the same basis of from 15 to 30% by weight. The proportion by weight accounted for by Michael acrylic acid oligomers having four or five acrylic acid molecules in condensed form is, on the same basis, normally in each case from about 5 to 20% by weight. The probability of formation of even higher Michael acrylic acid oligomers generally falls with the number of acrylic acid molecules to be condensed to form them. The proportion by weight of Michael acrylic acid oligomers which comprise more than five acrylic acid molecules on the same basis as above is therefore typically in each case less than 5% by weight, frequently even in each case less than 1% by weight. The experimental determination of the proportions by weight accounted for by the different Michael acrylic acid oligomers is possible, for example by means of HPLC (high pressure liquid chromatography). The content in liquids F, for which the process according to the invention is particularly suitable, of Michael acrylic acid oligomers may be from 15 to 35% by weight, but also from 25 to 30% by weight.

The number-average molecular weight of the acrylic acid polymer present in liquids F to be treated in accordance with the invention (expressed as a multiple of the weight of a hydrogen atom) is usually ≧500, frequently ≧750 and in many cases ≧1000. In general, it will, however, be ≦10⁶, frequently ≦750 000, often ≦100 000. In many cases, the aforementioned number-average molecular weight will be in the range from 50 000 to 150 000. The aforementioned molecular weight data are based on determinations by means of GPC (gel permeation chromatography).

The acrylic acid polymer may consist either of individual uncrosslinked linear polymer chains or of shorter, highly crosslinked polymer chains, but also of mixtures of the two aforementioned variants. Liquids F for which the process according to the invention is particularly suitable may comprise from 40 to 80% by weight, but also from 50 to 70% by weight, of acrylic acid polymer.

Useful polymerization inhibitors present in liquids F to be treated in accordance with the invention may, in principle, be all of those which are recommended in the prior art for the purpose of inhibiting free-radical polymerization of acrylic acid present in the liquid phase. The group of such polymerization inhibitors includes, for example, alkylphenols, for example o-, m- or p-cresol (methylphenol), 2-tert-butyl-4-methylphenol, 6-tert-butyl-2,4-dimethylphenol, 2,6-di-tert-butyl-4-methylphenol, 2-tert-butylphenol, 4-tert-butylphenol, 2,4-di-tert-butylphenol, 2-methyl-4-tert-butylphenol, 4-tert-butyl-2,6-dimethylphenol or 2,2′-methylenebis(6-tert-butyl-4-methylphenol), hydroxyphenols, for example hydroquinone, 2-methylhydroquinone, 2,5-di-tert-butylhydroquinone, pyrocatechol (1,2-dihydroxybenzene) or benzoquinone, aminophenols, for example para-aminophenol, methoxyphenol (guaiacol, pyrocatechol monomethyl ether), 2-ethoxyphenol, 2-isopropoxyphenol, 4-methoxyphenol (hydroquinone monomethyl ether), mono- or di-tert-butyl-4-methoxyphenol, tocopherols, for example o-tocopherol and 2,3-dihydro-2,2-dimethyl-7-hydroxybenzofuran (2,2-dimethyl-7-hydroxycoumaran), N-oxyls such as hydroxy-2,2,6,6-tetramethylpiperidine N-oxyl, 4-oxo-2,2,6,6-tetramethylpiperidine N-oxyl, 4-acetoxy-2,2,6,6-tetramethylpiperidine N-oxyl, 2,2,6,6-tetramethylpiperidine N-oxyl, 4,4′,4″-tris(2,2,6,6-tetramethylpiperidine N-oxyl) phosphite or 3-oxo-2,2,5,5-tetramethylpyrrolidone N-oxyl, aromatic amines or phenylenediamines for example, N,N-diphenylamine, N-nitrosodiphenylamine and N,N′-dialkyl-para-phenylenediamine, where the alkyl radicals may be the same or different and each independently consist of from 1 to 4 carbon atoms and may be straight-chain or branched, hydroxylamines, for example N,N-diethylhydroxylamine, phosphorus compounds, for example triphenylphosphine, triphenyl phosphite, hypophosphorous acid or triethyl phosphite, sulfur compounds, for example diphenyl sulfide or phenothiazine, if appropriate in combination with metal salts, for example the chlorides, dithiocarbamates, sulfates, salicylates or acetates of copper, manganese, cerium, nickel or chromium.

Useful inhibitors also include conversion products of the aforementioned compounds which are formed therefrom under the action of heat and/or oxidizing agent.

It will be appreciated that it is also possible to use different mixtures of all polymerization inhibitors mentioned. Preferably, the liquid F to be treated in accordance with the invention comprises phenothiazine and/or hydroquinone monomethyl ether as the polymerization inhibitor.

In general, the liquid F comprises, based on its weight, at least 10 ppm by weight, frequently at least 50 ppm by weight and in many cases at least 150 ppm by weight of polymerization inhibitor. The process according to the invention is particularly relevant in the case of liquids F, whose content of polymerization inhibitor on the same basis is ≦1% by weight, or ≦0.5% by weight.

The content of monomeric acrylic acid in liquids F to be treated in accordance with the invention is generally ≧5% by weight (based on the weight of the liquid F). The process according to the invention is particularly relevant in the case of liquids F whose content of monomeric acrylic acid is from ≧5 to ≦20% by weight, or from ≧10 to ≦20% by weight.

The other compounds present in liquids F to be treated in accordance with the invention are primarily compounds having a higher boiling point than acrylic acid at standard pressure. These include in particular fumaric acid, maleic acid and phthalic acid and the anhydrides thereof. Overall, the total amount of the aforementioned carboxylic acids and/or carboxylic anhydrides having a higher boiling point than acrylic acid, based on the weight of the liquid F, is generally ≦10% by weight, usually ≦5% by weight, but frequently ≧1% by weight, or ≧2% by weight.

However, it will be appreciated that the liquid F when performing the process according to the invention, as other compounds, may also comprise added active compounds whose presence reduces the formation of fouling. Useful such active compounds include, for example surfactants, as recommended by EP-A 1062197. In a corresponding manner, U.S. Pat. No. 3,271,296 recommends the addition of reaction products of propylenediamine with alkyl-alkenyl substituted succinic carboxylic acids to which dispersing action is ascribed (e.g. Komad® 313 from Mol (Hungary)). GB Patent No. 922 831 discloses correspondingly suitable active compounds.

Useful such active compounds for addition to the liquid F for the inventive purposes also include the nitrogen-comprising compounds recommended in German application 102006062258.8 (e.g. tertiary amines, salts formed from a tertiary amine and a Brønsted acid and/or quaternary ammonium compounds). Particularly advantageously, useful such added active compounds present in a liquid F are trimethylamine, triethylamine, N,N,N′,N′-tetramethyl-1,3-propanediamine and pentamethyldiethylenetriamine. The use amounts of such active compounds are, based on the weight of the liquid F, appropriately in application terms, from 0.5 to 1% by weight. In principle, however, use amounts on a corresponding basis of from 0.1 to 10% by weight are also possible. Of course, a liquid F to be treated in accordance with the invention may also comprise as added catalysts for redissociation of Michael acrylic acid oligomers, the active compounds recommended for this purpose in WO 2004/035514. Most preferably, a liquid F on entry into the at least one secondary space comprises those added active compounds (which may be selected, for example, from the aforementioned) which lower the interface tension in the liquid F for the formation of a gas bubble in the liquid F and hence facilitate the formation of gas bubbles which is important in accordance with the invention. Of course, a liquid F may also comprise a wide variety of different added mixtures of the aforementioned active compounds. Normally, liquids F comprise at least 1% by weight of other compounds (other than monomeric acrylic acid, acrylic acid polymer, Michael acrylic acid oligomers and polymerization inhibitors).

The total amount of Michael acrylic acid oligomers and acrylic acid polymer present in the liquid F will, based on the total amount of the liquid F, regularly be ≧60% by weight, in many cases ≧70% by weight and often ≧80% by weight.

Liquids F for which the process according to the invention is suitable accordingly comprise especially liquids F which comprise:
5 to 50% by weight of Michael acrylic acid oligomers,
40 to 80% by weight of acrylic acid polymer,
5 to 20% by weight of monomeric acrylic acid,
0.1 to 2% by weight of polymerization inhibitor, and
1 to 15% by weight other compounds (other than the aforementioned compounds).

However, liquids F for which the process according to the invention is suitable also comprise liquids F which comprise:
10 to 40% by weight of Michael acrylic acid oligomers,
50 to 70% by weight of acrylic acid polymer,
5 to 15% by weight of monomeric acrylic acid,
0.1 to 1% by weight of polymerization inhibitor, and
1 to 15% by weight of other compounds.

However, liquids F for which the process according to the invention is suitable also comprise liquids F which comprise:
15 to 35% by weight of Michael acrylic acid oligomers,
50 to 70% by weight of acrylic acid polymer,
5 to 15% by weight of monomeric acrylic acid,
0.1 to 1% by weight of polymerization inhibitor, and
1 to 15% by weight of other compounds.

In all compositions for liquids F specified in this document, of the up to 15% by weight (or of the from 1 to 15% by weight) of other compounds, from 1 to 8 percent by weight, or from 2 to 6 percent by weight may be accounted for by the total amount of fumaric acid, maleic acid, phthalic acid and anhydrides thereof present in the liquid F.

Moreover, the process according to the invention is advantageous especially when the polymerization inhibitor in the compositions of the liquid F specified in this document is formed by phenothiazine and/or hydroquinone monomethyl ether. Moreover, the process according to the invention is particularly advantageous when the liquid F is a solution (for example a homogeneous or a colloidal solution). In principle, the term “liquid F” shall, however, also encompass a fluid “solid-in-liquid dispersion”.

A characteristic feature of the process according to the invention is that, as the liquid F passes through the at least one secondary space of the indirect heat exchanger, liquid phase is maintained. In other words, the stream leaving the at least one secondary space of the indirect heat exchanger will at least partly be present in the liquid state. The liquid F, as it passes through the at least one secondary space of the indirect heat exchanger in the process according to the invention, is thus not converted fully to the gas phase. In general, in the process according to the invention, less than 50% by weight of the liquid F which flows in, as it flows through the at least one secondary space of the indirect heat exchanger, is converted to the gas phase (vapor phase; the terms “gaseous” and “vaporous” are used synonymously in this document). Frequently, this proportion by weight is even less than 30% by weight, and in many cases even less than 10% by weight.

One means of implementing the process according to the invention consists, for example, in that the liquid F comprises at least one added substance (which is encompassed among “other compounds”) whose boiling point at standard pressure (1 atm) is below (appropriately in application terms at least 10° C., better at least 20° C. and preferably at least 30° C. below the boiling point of acrylic acid; but generally not more than 60° C. below the boiling point of acrylic acid) that of acrylic acid. For example, useful such substances include water, aqueous solutions such as acid water, or other low molecular weight compounds which behave essentially inertly. Acid water is a further liquid phase obtained from the product gas mixture of the gas phase partial oxidation (employed for acrylic acid preparation). The term “acid water” firstly expresses the fact that the acid water comprises generally ≧50% by weight, frequently ≧60% by weight, in many cases ≧70% by weight and often ≧80% by weight of water. This is generally both water of reaction (i.e. water formed as a by-product of the gas phase partial oxidation), and diluent water (steam) used as part of the inert diluent gas in the gas phase partial oxidation. However, it also expresses the fact that the acid water, as well as water, also comprises small amounts of acrylic acid and secondary component acids, for example propionic acid, acetic acid and formic acid, and hence has a pH of <7 (the total content of the secondary component carboxylic acids other than acrylic acid is generally, based on the weight of the acid water, at values of ≦10% by weight, in some cases at values of ≦5% by weight). The acrylic acid content of the acid water will normally be from 2 to 15, or from 5 to 15, and frequently approx. 10% by weight (German application 102007055086.5, DE-A 102 43 625, WO 2004/035514 and DE-A 103 32 758 give specific details of the acid water formation by way of illustration).

For example acid water may comprise:
81.9% by weight of water,
9.4% by weight of acrylic acid,
4.1% by weight of acetic acid,
3.8% by weight of formaldehyde,
0.7% by weight of formic acid, and
0.01% by weight of propionic acid.

In general, acid water additionally comprises a small amount of polymerization inhibitor (e.g. MEHQ).

For example, the liquid F, based on its weight, may comprise up to 10% by weight, preferably in accordance with the invention up to 5% by weight, of at least one such added auxiliary substance having a lower boiling point than acrylic acid (such as water, acid water or other aqueous solutions). In general, the liquid F, based on its weight, will comprise at least 0.1% by weight, frequently at least 0.3% by weight, or at least 0.5% by weight, and in many cases at least 1% by weight, of at least one such added auxiliary substance having a lower boiling point than acrylic acid (such as water, acid water or other aqueous solutions). As the liquid F flows through the at least one secondary space, the pressure conditions are, appropriately in application terms, then adjusted such that the auxiliary substance evaporates at least partly in the at least one secondary space during the residence time of the liquid F, as a result of which the gas bubbles required in accordance with the invention are formed. When the liquid F is forcibly conveyed through the at least one secondary space of the heat exchanger by means of a pump, it is appropriate in application terms to meter the aforementioned auxiliary substance having a lower boiling point than acrylic acid into the liquid F between the pump outlet and entrance into the at least one secondary space.

Alternatively and/or simultaneously, an (auxiliary) substance which is gaseous both under the conditions existing within the at least one secondary space and before entry of the liquid F into the at least one secondary space can be metered into the liquid F just (for example immediately) upstream of its entry into the at least one secondary space of the heat exchanger, the presence of which substance then ensures the formation of the gas bubbles required in accordance with the invention in the at least one secondary space as it flows through the at least one secondary space. In principle, such a gaseous substance can also be metered only directly into the at least one secondary space or, in addition to the advance metered addition already described, also be metered into the at least one secondary space.

Both the liquid and the gaseous auxiliary substance is supplied to the liquid F, appropriately in accordance with the invention, at a temperature which essentially corresponds to that of the liquid F before entry thereof into the at least one secondary space of the indirect heat exchanger (the desired state of matter can be established by appropriate pressure adjustment).

In quite general terms, in accordance with the invention, forced conveying of the liquid F through the at least one secondary space is preferred. This is normally accomplished by means of a pump. In this case, the metered addition addressed above of a gaseous substance into the liquid F, advantageously in accordance with the invention, is effected along the conveying zone disposed between the outlet from the conveying pump and the inlet into the at least one secondary space (advantageously in accordance with the invention, as close as possible to the inlet into the at least one secondary space of the heat exchanger). Gaseous (auxiliary) substances suitable in accordance with the invention are, for example, those which are present in the gaseous state at standard pressure (1 atm) and temperatures above −40° C. Among these preference is given in accordance with the invention in turn to those whose constituents are also an element of the product (gas) mixture of the reaction employed for the preparation of the acrylic acid (for example of the heterogeneously catalyzed partial oxidation). In other words, useful such auxiliary substances to be metered in in gaseous form (auxiliary gases) especially include carbon oxides (CO₂, CO), noble gases such as He, Ar and Ne, molecular oxygen, molecular hydrogen, molecular nitrogen, but also mixtures of individual auxiliary gases already mentioned, for example air or lean air (the latter is oxygen-depleted air (i.e. it comprises essentially mixtures of molecular nitrogen and molecular oxygen, whose proportion by volume of molecular oxygen is lower than that of air)). It is particularly appropriate from an application point of view to use lean air or mixtures of molecular oxygen and molecular nitrogen, whose proportion by volume of molecular oxygen is from 1 to 15% by volume, or from 2 to 12% by volume, or from 4 to 10% by volume, or from 6 to 10% by volume, for example 8% by volume. A further auxiliary gas which is particularly advantageous in accordance with the invention is residual gas.

In this document, residual gas (the term “offgas” is also used in some cases) is understood to mean that gas mixture which remains when the acrylic acid and, if appropriate, acid water have been removed from the product gas mixture of the heterogeneously catalyzed gas phase partial oxidation carried out to prepare acrylic acid.

A portion of the residual gas is typically also used as “cycle gas” to dilute the reactants in the reaction gas mixture used for the gas phase partial oxidation (cf. for example, in German application 102007055086.5, WO 2004/035514, DE-A 10332758, DE-A 103 36 386).

Based on its total amount, residual gas comprises typically (for example in the case of a two-stage gas phase partial oxidation of propene to acrylic acid) the following constituents:
80 to 95% by wt. of molecular nitrogen,
2 to 15% by wt. of molecular oxygen,
2 to 10% by wt. of carbon oxides,
0.5 to 5% by wt. of water,
0.01 to 0.2% by wt. of acetic acid,
0.01 to 0.2% by wt. of acrylic acid,
0.01 to 0.5% by wt. of acrolein,
0.001 to 0.01% by wt. of propane and
0.1 to 2% by wt. of propylene.

A typical residual gas composition (for example in the case of a two-stage gas phase partial oxidation of propene to acrylic acid) may also be:
0.2651% by wt. of acrylic acid,
0.0989% by wt. of acetic acid,
2.9333% by wt. of water,
0.0059% by wt. of formic acid,
0.1720% by wt. of acrolein,
0.0002% by wt. of propionic acid,
0.0002% by wt. of furfurals
0.0013% by wt. of allyl formate,
4.7235% by wt. of molecular oxygen,
2.1171% by wt. of CO₂,
0.6921% by wt. of Co,
0.6466% by wt. of propane,
0.3161% by wt. of propylene and
88.0277% by wt. of molecular nitrogen.

It is favorable in accordance with the invention when, in the course of performance of the process according to the invention, based on the total (internal) volume of the at least one secondary space flowed through by the liquid F, from 0.1 or 0.5 to 25% by volume, preferably from 0.1 or 0.5 to 20% by volume and more preferably from 0.1 or 0.5 to 15 or to 10% by volume is accounted for by gas bubbles present therein (gas phase) and the particular remaining amount (i.e. from 75 to 99.9 or 99.5% by volume, preferably from 80 to 99.9 or 99.5% by volume and more preferably from 85 or 90 to 99.9 or 99.5% by volume) by liquid phase present therein (liquid) (excessively intense gas bubble formation (for example up to proportions by volume of ≧60% by volume) in the secondary space should be avoided in the process according to the invention, since it can reduce the heat transfer). Appropriate judgment of the volume flow of liquid F supplied to the at least one secondary space and volume flow of auxiliary gas simultaneously supplied thereto allows the aforementioned conditions to be established in a controlled manner. Instead of also using relatively low-boiling auxiliaries (or else accompanying them), the formation of gas bubbles required in accordance with the invention can also be promoted by establishing correspondingly lower working pressures in the at least one secondary space flowed through by the liquid F.

Advantageously in accordance with the invention, the liquid F flows into the at least one secondary space of the indirect heat exchanger with a temperature T^{F} of ≧150° C., better ≧160° C., preferably ≧165° C., more preferably ≧170° C., even more preferably ≧175° C., even better ≧180° C., further advantageously ≧185° C. and most advantageously ≧190° C. In general, the aforementioned T^{F} will, however, be ≦250° C., frequently ≦225° C., in many cases ≦200° C.

The difference ΔT^{A,F} between the temperature T^{F} and the temperature T^{A}, with which the heated substance mixture (the heated liquid) leaves the at least one secondary space of the heat exchanger again, will generally be at least 0.1° C., preferably at least 1° C., more preferably at least 2° C. and even better at least 5° C. Particularly advantageously, ΔT^{A,F} will be at least 10° C. Normally, ΔT^{A,F} will, however, be ≦100° C., frequently even ≦80° C. Typical ranges of values for ΔT^{A,F} are from 0.1 to 70° C., or to 50° C., or from 10 to 40° C., or to 30° C.

The working pressure of the liquid F on entry into the at least one secondary space is greater than on exit therefrom. Typical working pressure ranges for the at least one secondary space which are suitable for the inventive heat transfer are from 1 mbar to 10 bar, often from 10 mbar to 5 bar and in many cases from 50 mbar to 3 bar. Appropriately, in application terms, the pressure on the pressure side of the pump which conveys the liquid F through the at least one secondary space is from 3 to 10 bar, or from 4 to 6 bar.

In the case of an indirect heat exchanger for use in accordance with the invention, the heat is not transferred by direct contact between fluid heat carrier and liquid mixture to be heated which is forced by mixing. Instead, the heat is transferred indirectly between fluids separated by a dividing wall. The active separating area of the heat transferrer (heat exchanger) which is active for the heat transfer is referred to as heat exchange or transfer area, and the heat transfer follows the known laws of heat transfer.

It is essential to the invention that, in the process according to the invention, the indirect heat exchanger is flowed through both by the fluid heat carrier and by the liquid F. In other words, both flow into the heat exchanger and then back out again (one flows through the at least one primary space and the other through the at least one secondary space).

Useful fluid heat carriers for the process according to the invention are in principle all possible hot gases, vapors and liquids.

The primary fluid heat carrier is steam, which may be at different pressures and temperatures. Frequently, it is favorable when the steam condenses as it passes through the indirect heat exchanger (saturated steam).

Alternatively, useful fluid heat carriers include oils, melts, organic liquids and hot gases. Examples thereof are silicone compounds such as tetraaryl silicate, diphenyl-comprising mixture composed of 74% by weight of diphenyl ether and 26% by weight of diphenyl, chlorinated incombustible diphenyl, and also mineral oils and pressurized water.

The difference (T^{W}−T^{F}) between that temperature T^{W} with which the fluid heat carrier enters the at least one primary space of the heat exchanger in the course of performance of the process according to the invention and that temperature T^{F}, with which the liquid F enters the at least one secondary space of the same heat exchanger, may be, for example, from 1 to 150° C., frequently from 5 to 100° C., or from 10 to 80° C., in many cases from 20 to 60° C., or from 15 to 30° C.

Indirect heat exchangers suitable for the process according to the invention are especially double tube, tube bundle, ribbed tube, spiral or plate heat transferrers. Double tube heat transferrers consist of two concentric tubes. A plurality of these double tubes can be joined to form tube walls. The inner tube may be smooth or be provided with ribs to improve the heat transfer. In individual cases, a tube bundle can also replace the inner tube. The fluids exchanging heat may move in cocurrent or in countercurrent. Appropriately in accordance with the invention, the liquid F is conveyed upward in the inner tube and hot steam flows downward, for example in the ring space.

For the process according to the invention, tube bundle heat transferrers are particularly suitable. They normally consist of a closed wide outer tube which encloses the numerous smooth or ribbed transferrer tubes of small diameter, which are secured to the tube plates.

The distances from tube center to tube center of the bundle tubes is, appropriately in application terms, from 1.3 to 2.5 times the outer tube diameter. The large specific heat exchange area which arises—as the exchange area per unit of space required—is an advantage of the tube bundle heat transferrer. The vertical or horizontal tube bundle heat transferrers differ, inter alia, in the tube design. The transferrer tubes may be straight, bent in a U shape or else designed as a multiflow spiral tube bundle.

The liquid F to be heated in accordance with the invention flows, in accordance with the invention, preferably within the transferrer tubes (in principle, it may, though also flow within the space surrounding the transferrer tubes, and the heat carrier within the transferrer tubes). The fluid heat carrier (preferably saturated water vapor), appropriately in accordance with the invention, flows outside the transferrer tubes.

Guide plates for better conduct of the fluid heat carrier in the outer space are appropriate in accordance with the invention and generally serve the additional purpose of supporting the transferrer tubes. The guide plates generally increase the flow rates in the outer space and thus the heat transfer coefficients, among other parameters. The flow in the outer space advantageously runs transverse to the transferrer tubes. According to the flow direction of the outer space fluid in relation to the transferrer tubes, it is possible to distinguish, for example, longitudinal flow and crossflow and transverse flow tube bundle heat transferrers. In principle, the fluid heat carrier may also be moved in a meandering manner around the transferrer tubes and be conducted in cocurrent or countercurrent to the liquid mixture to be heated in accordance with the invention only viewed over the tube bundle heat exchanger. Spiral tube bundle heat transferrers also generally utilize the advantages of crossflow. The tubes alternate—from position to position—between right- and left-handed spirals. The outer space fluid flows in countercurrent to the tube fluid and flows around the spiral tubes in crosscurrent.

In the single-flow tube bundle heat transferrer, the liquid F to be heated in accordance with the invention moves through all transferrer tubes in the same direction.

Multiflow tube bundle heat transferrers comprise tube bundles subdivided into individual sections (the individual sections generally comprise an identical number of tubes). Dividing walls divide the chambers adjoining the tube plates (through which the transferrer tubes are conducted with sealing and to which they are secured) into sections and deflect the liquid F entering the chamber part from one section into a second section and thus back. According to the number of sections, the liquid F to be heated in accordance with the invention flows through the length of the tube bundle heat transferrer more than once (twice, three times, four times, etc.) with high velocity in alternating direction (two-flow, three-flow, four-flow, etc. tube bundle heat transferrers). Heat transfer coefficient and exchange length increase correspondingly.

Plate heat transferrers (plate heat exchangers) are normally constructed in a compact design, in the manner of filter presses, generally from corrugated or otherwise profiled plates provided with channels for the fluid heat carrier and the liquid mixture to be heated (generally composed of graphite or metal, for example stainless steel). The two heat-exchanging fluids then flow in cocurrent, countercurrent and/or crosscurrent alternating as thin layers (for example upward and downward) through their chamber series and transfer heat to one another at both chamber walls. The corrugated plate profiles increase the turbulence and improve the heat transfer coefficients. Plate heat exchangers suitable for the inventive purpose are, for example, described in EP-A 107 9194, U.S. Pat. No. 6,382,313, EP-A 123 2004 and WO 01/32301. Tube bundle heat exchangers are for example described in EP-A 700 893, EP-A 700 714 and DE-A 443 1949. Spiral and ribbed tube heat exchangers are described, for example, in Vauck/Müller, Grundoperationen chemischer Verfahrenstechnik [Basic Operations in Chemical Process Technology], 4th edition, Verlag Theodor Steinkopf, Dresden (1974) and in Ullmanns Encyclopädie der technischen Chemie, Volume 2, Verfahrenstechnik I (Grundoperationen) [Process Technology I (Basic Operations)], 4th edition, 1972, p. 432 ff.

As already mentioned, it is particularly advantageous in accordance with the invention when the liquid F is forcibly conveyed through the at least one secondary space of the indirect heat exchanger, for example with the aid of a pump. Preferably, in accordance with the invention the process according to the invention will therefore be carried out by using forced-circulation tubular heat exchangers (forced-circulation tube bundle heat transferrers).

Preferably, the liquid F is forcibly conveyed into the tubes thereof.

For example, the process according to the invention can be performed by using a three-flow tube bundle heat transferrer, through whose tubes the liquid F is forcibly conveyed.

In other words, the tube interiors form the secondary spaces of the heat exchanger. The outer tube diameter may be 38 mm, with a wall thickness of the tubes of 2 mm. At a length of the tubes of 4800 mm, their total number is, appropriately in application terms, 234 (in each case 78 tubes for one flow direction). The tube pitch is simultaneously advantageously 48 mm (300 distribution). 9 deflecting plates (plate thickness: in each case 5 mm) mounted between the tube plates (in which the exchanger tubes are secured) divide the cylindrical space surrounding the heat transferrer tubes (primary space) into 10 longitudinal sections (segments). All 9 deflecting plates are in principle circular. The circle diameter is 859 mm. On each of the circular deflecting plates, however, a half-moon-shaped circle segment is cut out, whose area is 35.8% of the total area, so as to form an appropriate passage for steam as the heat carrier, these passages being mounted opposite one another in alternating succession (otherwise, the deflecting plates are secured with sealing at the vessel wall; where the heat transferrer tubes meet the deflecting plates, there are appropriate bores in the deflecting plates). Appropriately in application terms, steam is conducted as a heat carrier through the space surrounding the heat transferrer tubes. The entry of steam and liquid F into the three-flow tube bundle heat transferrer is, favorably in application terms, disposed on the same side of the heat transferrer (later in this patent application, the above-described heat transferrer is referred to as three-flow tube bundle heat transferrer D*). The pump used to convey the liquid F is appropriately a centrifugal pump (preferably with a stepped impeller) with a double-action slip ring seal according to DE-A 102 28 859, the barrier liquid preferably being a water/glycol mixture. The working pressure on the pressure side of the pump (before entry into the at least one secondary space of the heat exchanger) is advantageously from 4 to 6 bar (unless explicitly stated otherwise, should always be understood as absolute pressure (barabs)) and more preferably 6 bar. The circulation rate of liquid F to be heated in accordance with the invention is typically from 100 to 700 m³/h, advantageously from 300 to 500 m³/h.

Alternatively, for the process according to the invention, it is also possible to use a thirteen-flow tube bundle heat transferrer through whose tubes the liquid F is forcibly conveyed. Advantageously, in accordance with the invention, the cylinder which surrounds the primary space is equipped with a compensator (dimensions of compensator: diameter=2.075 m; height=670 mm; 3 bellows; installation site at half the height of the vertically aligned primary space), which enables the low-tension thermal expansion of the apparatus in the course of heating and cooling.

The outer tube diameter may again be 38 mm with a wall thickness of the tubes of 2 mm. At a length of the tubes of 5000 mm their total number is, appropriately in application terms, 1066 (in each case 82 tubes for one flow direction). The tube pitch is simultaneously advantageously 47 mm (60° distribution). 9 deflecting plates (plate thickness: in each case 10 mm) mounted between the tube plates (in which the exchanger tubes are secured) divide the cylindrical space (primary space) surrounding the heat transferrer tubes into 9 longitudinal sections (segments). All 9 deflecting plates are in principle circular. The circle diameter is 1734 mm. On each of the circular deflecting plates, however, a half-moon-shaped circle segment is cut out, whose area is 15% of the total area, so as to form an appropriate passage for steam as the heat carrier, these passages being mounted opposite one another in alternating succession (otherwise, the deflecting plates are secured with sealing at the vessel wall; where the heat transferrer tubes meet the deflecting plates, there are appropriate bores in the deflecting plates). Appropriately, in application terms, steam is conducted as a heat carrier through the space surrounding the heat transferrer tubes. The entry of steam and liquid F into the thirteen-flow tube bundle heat transferrer is, favorably in application terms, disposed on the same side of the heat transferrer. The pump used to convey the liquid F is appropriately a centrifugal pump (preferably with a stepped impellor) with a double-action slip ring seal according to DE-A 102 28 859, the barrier liquid preferably being a water/glycol mixture. The working pressure on the pressure side of the pump (before entry into the at least one secondary space of the heat exchanger) is advantageously from 4 to 6 bar, particularly advantageously 6 bar.

The circulation rate of liquid F to be heated in accordance with the invention is typically from 100 to 600 m³/h, frequently from 100 to 250 m³/h. The connection of conveying pump and tube bundle heat transferrer is, in this sequence toward the heat transferrer, appropriately in application terms, in succession, 1 DN 200 bend (internal diameter=267 mm), 7 DN 300 pipe bends (internal diameter=317 mm) and 1 DN 300 pressure side pipe.

Quite generally, it is advantageous in accordance with the invention when the tubes of a tube bundle heat transferrer used in accordance with the invention are internally and/or externally structured heat exchanger tubes. Such heat exchanger tubes are obtainable, for example, from Wieland-Werke AG in D-89070 Ulm, and are described, for example in its patents EP-A 1 158 268, EP-A 1 113 237, EP-A 1 182 416, EP-A 1 830 151 and EP-A 1 223 400. Internally structured heat exchanger tubes are advantageous in accordance with the invention in particular when the tube interior in the process according to the invention forms the at least one secondary space, since the internal structuring increases the number of nucleation sites for the formation of gas bubbles (for example in the case of nucleate boiling).

Alternatively to the aforementioned inner structuring of heat exchanger tubes to be used in accordance with the invention or in addition thereto, it is advantageously possible in accordance with the invention to insert so-called turbulence generators (turbulators) into the interior of the heat exchanger tubes, as described, for example, in EP-A 1 486 749 and the prior art cited in this document.

Turbulence elements (turbulence generators) which are particularly advantageous in accordance with the invention are manufactured from wires or thin rods with a round cross section and are sold, for example, by CalGavin on their Internet home page http://www.calgavin.co.uk/HITRAN/hitran.htm// (Apr. 17, 2008) under the name HITRAN® Thermal System.

These turbulators are also described in
Fluiddynamik in a tube equipped with wire matrix inserts, Alex Smeethe, PeterDroegenmuleler, Waldemar Bujalski, Joe Wood, CHISA 2004, 16th INTERNATIONAL CONGRESS OF CHEMICAL AND PROCESS ENGINEERING, 22-26 Aug. 2004, Prague, Czech Republic;
Effect of hiTRAN Inserts on tube fouling at low Reynolds Number, International Research Project, SmaI, N.E., Anderson, K., Glass, D, University of Edinburgh, 2004; and
Use of ln-Tube to reduce Fouling from Crude Oils, B. D. Crittenden, S. T. Kolaczkowski, and T. Takemoto, AlChemE Symp Series Vol. 89 (No 295), Heat Transfer—Atlanta 1993, pp 300-307.

The reason for the success of the inventive procedure is probably that, under the thermal boundary conditions thereof, the Michael acrylic acid oligomers are redissociated at least partly to monomeric acrylic acid. In spite of the presence of polymerization inhibitors, locally elevated concentrations of uninhibited acrylic acid monomers form in this way. The acrylic acid polymer present dissolved in the liquid F additionally causes three-dimensional preferred alignments both of the Michael acrylic acid oligomers and of the resulting monomeric acrylic acid owing to corresponding hydrogen bond formation. In the absence of a mechanism which causes rapid conveying of the monomeric acrylic acid formed from the liquid phase into the gas phase, the acrylic acid polymer already present thus brings about catalytic acceleration of the undesired free-radical polymerization of the acrylic acid monomers formed.

In addition to the formation of gas bubbles which effectively strip the monomeric acrylic acid formed out of the liquid phase, or alternatively thereto, the liquid F can also be conveyed through the at least one secondary space distributed as a liquid film over the entire heat exchange area (i.e. as a thin layer of liquid F adjoining a gas phase). Correspondingly designed heat transferrers are referred to as thin film heat transferrers. Thin film heat transferrers usable in accordance with the invention are falling film heat transferrers. The heat transfer can take place, for example in a tube in which the liquid F flows down on the tube inner wall as a coherent liquid film, while the heat carrier is conducted along the outer wall. For example the liquid F can be distributed uniformly over the upper tube plate of the heating chamber by means of nozzles or by a distributor system. The liquid F then flows downward under gravity as a thin film on the inner walls of the long heat transferrer tubes. Vapor bubbles leaving the liquid likewise flow downward and accelerate the flow rate of liquid film. Immediately below the heat transferrer tubes is normally arranged a separator.

The liquid-vapor mixture enters this separator, and internals separate the vapors from the remaining liquid phase. A centrifugal pump draws the latter off. Either only the vapors or both are then appropriately recycled into that separating column in which the liquid F has formed in the course of thermal separation of another acrylic acid-comprising liquid mixture. In principle, it is advantageously additionally possible in accordance with the invention to conduct a gas stream (for example air, lean air, nitrogen and/or residual gas) through the tube interior (in parallel or in countercurrent to the falling liquid film). FIG. 278 in “Grundoperationen chemischer Verfahrenstechnik, R. A. Vauck, H. A. Müller, Verlag Theodor Steinkopf, Dresden 1974 (page 558)”, shows a schematic diagram of a falling film heat transferrer suitable in accordance with the invention. Further falling film heat transferrers suitable in accordance with the invention are detailed in WO 08/010,237.

In the case of thin film heat transferrers, temperature differences between heat carrier and liquid F of from 3 to 8° C. are frequently sufficient. However, they may also be from 20 to 40° C. The residence time of the liquid F in the thin film heat transferrer is typically from 1 to 3 min.

Thin film heat transferrers (those used for the process according to the invention may also be Sambay or Lura evaporators or filmtruders) are likewise heat transferrers with forced conveying of the liquid to be heated. In the case of falling film heat transferrers, the forced conveying is brought about by gravity. In rotor heat transferrers, the liquid film is generated by a rotor system on a vertical axis, which is driven by an external motor. The rotor system is disposed in the tube interior in which the liquid F is introduced.

In centrifugal heat transferrers, the centrifugal force distributes the liquid F flowing in from the top as a turbulently flowing film on to hot, rotating internals. The vapor is formed in the space of seconds and the liquid concentrate spins separately from the vapors into a collecting channel.

Typical values for the film thickness of the liquid film in thin film evaporators are from 0.1 to 2 mm.

Quite generally, the process according to the invention is performed in a thin film heat transferrer, preferably under reduced pressure. Appropriately, in accordance with the invention, the pressure employed is ≦0.5 atm, preferably from 0.01 to 0.5 atm, more preferably from 0.01 to 0.3 atm and most preferably from 0.01 to 0.1 atm.

Further thin film evaporators which can be used for the process according to the invention are advantageously helical tube evaporators.

In their coiled-pipe design, they are described, for example, in Chemie-Ing. Techn., 42, 1970/6, page 349 to 354.

This variant (especially according to FIG. 1 of the above document) is very particularly preferred in accordance with the invention.

Instead of a simple pipe design, it is also possible to employ an embodiment with two tubes arranged one inside the other, as disclosed, for example, in DE-A 1667051.

In the case of use of a helical tube evaporator, it is very particularly advantageous in accordance with the invention to meter a gaseous auxiliary substance (a gas stream, for example lean air or residual gas) into the liquid F before it enters the helical tube evaporator.

This achieves rapid establishment of the annular flow in the helical tube and hence an improvement in the heat and mass transfer.

The process according to the invention is, for example, relevant for a process in which the conversion of acrylic acid from the product gas mixture of a heterogeneously catalyzed gas phase partial oxidation of at least one C₃ precursor compound of acrylic acid (e.g. propylene, acrolein and/or propane) at elevated temperature with molecular oxygen over solid-state catalysts into the liquid phase is effected, for example, by passing the acrylic acid-comprising product gas mixture, if appropriate after indirect and/or direct cooling thereof, into a condensation column equipped with separating internals (preferably mass transfer trays) and allowing it to ascend into itself within the condensation column, fractionally condensing as it does so, and withdrawing crude acrylic acid from the condensation column, in the side draw, whose acrylic acid content is generally ≧90% by weight, in many cases even ≧95% by weight (cf., for example German application 102006062258.8, German application 102007055086.5, DE-A 102 35 847, WO 200/53560, DE-A 102 43 625, WO 2004/035514 and DE-A 103 32 758). The thermal energy required for this separation of the product gas mixture of the gas phase partial oxidation is essentially already provided by the hot product gas mixture.

As an outlet for the secondary components having a higher boiling point than acrylic acid, bottoms liquid comprising these secondary components, or, via a side draw disposed below the side draw for the crude acrylic acid, high boiler fraction comprising these secondary components, or a mixture of such bottoms liquid and high boiler fraction is withdrawn from the bottom of the condensation column (all referred to hereinafter collectively as high boiler liquid). A portion of the aforementioned high boiler liquid can be used to directly cool the product gas mixture of the gas phase partial oxidation and, by means of this direct cooling in the high boiler region of the condensation column, be recycled into it.

The high boiler liquid which has been withdrawn from the condensation column but not recycled into the condensation column by this route still comprises significant amounts of acrylic acid. In order to prevent this acrylic acid from being sent to disposal together with the high-boiling secondary components (i.e. in order to increase the yield of acrylic acid), the high boiler liquid is therefore advantageously subjected before this disposal to a stripping at elevated temperature. The stripping gas used is appropriately some of the residual gas which leaves the condensation column at the top thereof, and in particular comprises the constituents of the product gas mixture of the gas phase partial oxidation which are the most difficult to condense. To this end, an appropriate portion thereof is appropriately compressed and superheated (generally to the temperature existing in the bottom of the stripping column). The stripping itself is, advantageously in application terms, carried out in a rectification column (stripping column) comprising separating internals (preferably equidistant dual flow trays), in whose lower section (lower third of the theoretical plates) the high boiler liquid to be stripped is advantageously supplied.

In order to ensure very high stripping efficiency, in a manner appropriate to the aim, acrylic acid-comprising liquid is withdrawn continuously from the bottom of the stripping column, conducted through an indirect heat exchanger for the purpose of heating it and then predominantly conveyed back heated into the stripping column (preferably below the feed of the high boiler liquid to be stripped into the stripping column). The stripping gas is preferably fed to the bottom of the stripping column. The other portion of the bottoms liquid heated in the heat exchanger from the stripping column is conducted into a vessel under viscosity (preferred), density or temperature control, degassed therein, diluted with methanol and then sent to residue incineration. In the stripping column, a gas mixture comprising acrylic acid rises therein. In countercurrent above the feed point of the liquid to be stripped, reflux liquid is advantageously supplied, in order to ensure an increased separating action, especially with respect to the high-boiling secondary components whose boiling point is not very different than that of acrylic acid. To obtain the reflux liquid, the gas mixture which is conducted, for example, through a chimney tray which concludes the separating internals in the upper region of the stripping column, is cooled and partially condensed downstream thereof by direct cooling in a spray cooler.

The condensate is collected by the chimney tray, which simultaneously functions as a collecting tray and withdrawn therefrom. A portion is cooled in an indirect cooler and then recycled as cooling liquid for the direct spray cooling. For the purpose of inhibition of polymerization, a further amount of high boiler liquid which is to be stripped and comprises polymerization inhibitor is advantageously supplied to the portion or entirety of the condensate withdrawn for this purpose before it is cooled, and a portion of the resulting mixture, before entry thereof into the indirect cooler, is recycled into the stripping column essentially immediately below the chimney tray as reflux liquid. If required, a portion of condensate withdrawn from the chimney tray can also be recycled directly into the bottom of the condensation column.

The gas stream which has not been condensed in the course of spray cooling, leaves the stripping column in gaseous form and bears the acrylic acid stripped out is, appropriately in application terms, combined with the product gas mixture coming from the gas phase partial oxidation (preferably, for example, within the direct cooling thereof) or recycled into the bottom space of the condensation column (preferably not immersed). The amount of residual gas which leaves the condensation column and is not used for stripping is, if required, recycled partly as inert diluent gas into the heterogeneously catalyzed gas phase partial oxidation and the amount of residual gas not usable there is disposed of, for example, incinerated. Otherwise, the procedure may be as described, for example, in German application 102006062258.8 and in German application 102007055086.5.

When the above-described process is performed continuously over a prolonged period, a steady state is frequently attained, in which the liquid to be removed from the bottom of the stripping column and to be recycled heated into the stripping column is a liquid F to be treated in accordance with the invention. To heat it, the procedure will advantageously be that according to the invention. The indirect heat exchanger used will, appropriately in application terms, be a steam-heated tube bundle heat transferrer, and the gas bubbles required in accordance with the invention will be formed by supplying to the bottoms liquid withdrawn from the bottom of the stripping column, before it is forcibly conveyed through the secondary space of the heat transferrer (the interiors of its tubes) for example, an auxiliary gas (preferably the gas used for the stripping) or a low-boiling auxiliary liquid (preferably acid water) or both. The withdrawal of the bottoms liquid (of the liquid F) from the stripping column and its conveyance through the indirect heat transferrer will appropriately be undertaken as described in this document by means of a centrifugal pump. The liquid F heated in accordance with the invention can then, after it leaves the indirect heat transferrer, be recycled into the stripping column together with the auxiliary gas used or the auxiliary liquid used (or with auxiliary gas and auxiliary liquid) as described. As further assistants, the active compounds described at the outset of this document (e.g. surfactants, redissociation catalysts, tertiary amines, etc.) can be added to the bottoms liquid to be withdrawn from the bottom of the stripping column.

The process according to the invention is quite generally of significance when, in a thermal separating process, a liquid stream comprising acrylic acid is conducted for separation purposes into a separation column comprising separating internals, and the energy for a thermal separating process is supplied such that liquid is withdrawn from the separating column below the feed point of the stream to be treated with separating action, heated with the aid of an indirect heat exchanger and recycled thus heated into the separating column below the feed point of the stream to be treated with separating action, and the liquid withdrawn from the separating column is a liquid F.

The present invention thus comprises especially the following embodiments:
1. A process for transferring heat to a liquid F comprising dissolved monomeric acrylic acid, Michael acrylic acid oligomers and acrylic acid polymer with the aid of an indirect heat exchanger having at least one primary space and at least one secondary space separated from the at least one primary space by a material dividing wall D, in which the liquid F flows through the at least one secondary space, while the at least one primary space is simultaneously flowed through by a fluid heat carrier W, where the liquid F flows into the at least one secondary space with a temperature T^{F}≧130° C. and the fluid heat carrier W into the at least one primary space with a temperature T^{W}>T^{F}, wherein
   a) the liquid F on entry into the at least one secondary space comprises
      5 to 50% by weight of Michael acrylic acid oligomers,
      35 at least 40% by weight of acrylic acid polymer,
      up to 25% by weight of monomeric acrylic acid,
      up to 2% by weight of polymerization inhibitor, and
      up to 15% by weight of other compounds,
   and
   b) in the liquid F flowing through the at least one secondary space, during the flow through the at least one secondary space, the formation of gas bubbles and/or of thin layers of the liquid F adjoining a gas phase is brought about.
2. The process according to embodiment 1, wherein the liquid F on entry into the at least one secondary space comprises
   5 to 50% by weight of Michael acrylic acid oligomers,
   40 to 80% by weight of acrylic acid polymer, to 20% by weight of monomeric acrylic acid,
   0.1 to 2% by weight of polymerization inhibitor, and
   1 to 15% by weight of other compounds.
3. The process according to embodiment 1, wherein the liquid F on entry into the at least one secondary space
   10 to 40% by weight of Michael acrylic acid oligomers,
   50 to 70% by weight of acrylic acid polymer,
   5 to 15% by weight of monomeric acrylic acid,
   0.1 to 1% by weight of polymerization inhibitor, and
   1 to 15% by weight of other compounds.
4. The process according to embodiment 1, wherein the liquid F
   15 to 35% by weight of Michael acrylic acid oligomers,
   50 to 70% by weight of acrylic acid polymer,
   5 to 15% by weight of monomeric acrylic acid,
   0.1 to 1% by weight of polymerization inhibitor, and
   1 to 15% by weight of other compounds.
5. The process according to any one of embodiments 1 to 4, wherein the total amount of the Michael acrylic acid oligomers present in the liquid F on entry into the at least one secondary space consists of Michael acrylic acid dimers to an extent of from 40 to 60% by weight.
6. The process according to any one of embodiments 1 to 5, wherein the total amount of the Michael acrylic acid oligomers present in the liquid F on entry into the at least one secondary space consists of Michael acrylic acid trimers to an extent of from 15 to 30% by weight.
7. The process according to any one of embodiments 1 to 6, wherein the number-average molecular weight of the acrylic acid polymer present in the liquid F on entry into the at least one secondary space is from 500 to 10⁶.
8. The process according to any one of embodiments 1 to 6, wherein the number-average molecular weight of the acrylic acid polymer present in the liquid F on entry into the at least one secondary space is from 750 to 750 000.
9. The process according to any one of embodiments 1 to 6, wherein the number-average molecular weight of the acrylic acid polymer present in the liquid F on entry into the at least one secondary space is from 1000 to 100 000.
10. The process according to any one of embodiments 1 to 9, wherein the total amount of compounds from the group consisting of fumaric acid, maleic acid, phthalic acid and the anhydrides of these carboxylic acids present in the liquid F on entry into the at least one secondary space is ≦10% by weight.
11. The process according to any one of embodiments 1 to 10, wherein the liquid F on entry into the at least one secondary space comprises at least one added substance whose boiling point at standard pressure is below that of acrylic acid.
12. The process according to embodiment 11, wherein the liquid F comprises, as the added substance present, water and/or an aqueous solution.
13. The process according to embodiment 12, wherein the liquid F comprises, as the added substance present, acid water.
14. The process according to any one of embodiments 1 to 13, wherein at least one substance whose boiling point at 1 atm is ≦−40° C. has been metered into the liquid F before it enters the at least one secondary space.
15. The process according to any one of embodiments 1 to 13, wherein residual gas has been metered into the liquid F before it enters the at least one secondary space.
16. The process according to any one of embodiments 1 to 15, wherein 130° C.≦T^{F}≦250° C.
17. The process according to any one of embodiments 1 to 15, wherein 150° C.≦T^{F}≦225° C.
18. The process according to any one of embodiments 1 to 15, wherein 160° C.≦T^{F}≦200° C.
19. The process according to any one of embodiments 1 to 18, wherein the difference ΔT^{A,F} between the temperature T^{F} and the temperature T^{A} with which the heated liquid leaves the at least one secondary space is from 0.1 to 70° C.
20. The process according to any one of embodiments 1 to 18, wherein the difference ΔT^{A,F} between the temperature T^{F} and the temperature T^{A} with which the heated liquid leaves the at least one secondary space is from 5 to 50° C.
21. The process according to any one of embodiments 1 to 20, wherein T^{W}-T^{F} is from 1 to 150° C.
22. The process according to any one of embodiments 1 to 20, wherein T^{W}-T^{F} is from 5 to 100° C.
23. The process according to any one of embodiments 1 to 20, wherein T^{W}-T^{F} is from 20 to 60° C.
24. The process according to any one of embodiments 1 to 23, wherein, based on the total volume of the at least one secondary space flowed through by the liquid F, from 0.1 to 25% by volume is accounted for by gas bubbles present therein and from 99.9 to 75% by volume by liquid phase present therein.
25. The process according to any one of embodiments 1 to 23, wherein, based on the total volume of the at least one secondary space flowed through by the liquid F, from 0.5 to 15% by volume is accounted for by gas bubbles present therein and from 99.5 to 85% by volume by liquid phase present therein.
26. The process according to any one of embodiments 1 to 25, wherein the liquid F is forcibly conveyed through the at least one secondary space.
27. The process according to any one of embodiments 1 to 26, wherein the indirect heat exchanger is a tube bundle heat transferrer.
28. The process according to any one of embodiments 1 to 27, wherein the indirect heat exchanger is a multiflow tube bundle heat transferrer.
29. The process according to any one of embodiments 1 to 26, wherein the indirect heat exchanger is a plate heat transferrer.
30. The process according to any one of embodiments 1 to 26, wherein the indirect heat exchanger is a thin film heat transferrer.
31. The process according to any one of embodiments 1 to 26, wherein the indirect heat exchanger is a falling film heat transferrer.
32. The process according to any one of embodiments 1 to 26, wherein the indirect heat exchanger is a helical tube evaporator.
33. The process according to any one of embodiments 1 to 32, wherein the liquid F on entry into the at least one secondary space comprises at least one added active substance which lowers the interface tension for the formation of a gas bubble in the liquid F.

### EXAMPLES AND COMPARATIVE EXAMPLES

### Comparative example 1

The product gas mixture of a two-stage heterogeneously catalyzed partial gas phase oxidation of propylene (chemical grade) to acrylic acid was subjected to a fractional condensation in order to remove the acrylic acid present in the product gas mixture therefrom.

From the bottom region of the condensation column, high boiler liquid was withdrawn, which still comprised significant amounts of recoverable acrylic acid.

For the purpose of recovering it, the bottoms liquid was fed to a stripping column. The stripping gas used was compressed residual gas conducted out of the condensation column. The energy was introduced with the aid of a forced-circulation flash evaporator. At the steady state, a liquid F was withdrawn from the bottom of the stripping column and comprised:

| | |
|---|---|
| monomeric acrylic acid | 9.30% by wt. |
| Michael diacrylic acid (2 AA) | 10.62% by wt. |
| Michael triacrylic acid (3 AA) | 5.19% by wt. |
| 4 AA | 2.69% by wt. |
| 5 AA | 3.38% by wt. |
| 6 AA | 0.10% by wt. |
| 7 AA | 0.10% by wt. |
| 8 AA | 0.20% by wt. |
| 9 AA | 0.10% by wt. |
| 10 AA | 0.10% by wt. |
| acrylic acid polymer | 63.14% by wt. |
| fumaric acid | 2.00% by wt. |
| maleic acid | 1.96% by wt. |
| phthalic acid | 0.78% by wt., and |
| | PTZ, MEHQ and conversion products |
| | together were the remainder |
| | to 100% by weight. |

204.3 g of liquid F were initially charged in a 250 ml four-neck flask with a distillation apparatus attached. The distillation apparatus was cooled by means of flowing water whose temperature was kept constant in the range from 15 to 20° C. and the condensate formed therein was fed to a receiver flask.

To simulate the conditions in a circulation heat transferrer, the liquid F was circulated continuously in the four-neck flask with the aid of a magnetic stirrer. With the aid of an oil bath, the liquid F present in the four-neck flask was heated at standard pressure to a heating temperature of 170° C.

The pressure in the four-neck flask was then reduced to 290 mbar.

These conditions (internal temperature 170° C., 290 mbar, circulation) were maintained over a period of 3 h. Over the entire treatment period, no gas bubbles formed in the liquid F. At the end of the 3-hour treatment, the liquid present in the four-neck flask had the following composition:

| | |
|---|---|
| monomeric acrylic acid | 7.93% by wt. |
| 2 AA | 5.76% by wt. |
| 3 AA | 3.59% by wt. |
| 4 AA | 2.31% by wt. |
| 5 AA | 2.66% by wt. |
| 6 AA | 0.10% by wt. |
| 7 AA | 0.39% by wt. |
| 8 AA | 0.05% by wt. |
| 9 AA | 0.19% by wt. |
| 10 AA | 0.29% by wt. |
| acrylic acid polymer | 71.49% by wt. |
| fumaric acid, | 2.02% by wt. |
| maleic acid | 1.97% by wt. |
| phthalic acid | 0.79% by wt., and |
| | PTZ, MEHQ and conversion products |
| | together were the remainder |
| | to 100% by weight. |

The total amount of liquid still present in the four-neck flask was 203.1 g. In the distillation apparatus 1.2 g of monomeric acrylic acid had condensed (purity >99% by weight).

### Comparative Example 2

Comparative example 1 was repeated with the same liquid F, but the heating temperature selected was 180° C. In addition, the initial weight of liquid F was 207.0 g. Over the entire treatment, no formation of gas bubbles was observable. At the end of the 3-hour treatment, the liquid present in the four-neck flask had the following composition:

| | |
|---|---|
| monomeric acrylic acid | 5.47% by wt. |
| 2 AA | 4.06% by wt. |
| 3 AA | 2.76% by wt. |
| 4 AA | 1.57% by wt. |
| 5 AA | 1.79% by wt. |
| 6 AA | 0.11% by wt. |
| 7 AA | 0.27% by wt. |
| 8 AA | 0.05% by wt. |
| 9 AA | 0.11% by wt. |
| 10 AA | 0.16% by wt. |
| acrylic acid polymer | 77.7% by wt. |
| fumaric acid | 2.22% by wt. |
| maleic acid | 2.22% by wt. |
| phthalic acid | 0.92% by wt., and |
| | PTZ, MEHQ and conversion products |
| | together were the remainder |
| | to 100% by weight. |

The total amount of liquid still present in the four-neck flask was 184.6 g. In the distillation apparatus 22.4 g of monomeric acrylic acid had condensed (purity >99% by weight).

### Comparative Example 3

Comparative example 1 was repeated. The heating temperature selected was, however, 190° C. In addition, the composition of the liquid F with an initial weight of 214.1 g was as follows:

| | |
|---|---|
| monomeric acrylic acid | 10.30% by wt. |
| 2 AA | 14.29% by wt. |
| 3 AA | 6.21% by wt. |
| 4 AA | 3.08% by wt. |
| 5 AA | 3.50% by wt. |
| 6 AA | 0.19% by wt. |
| 7 AA | 0.42% by wt. |
| 8 AA | 0% by wt. |
| 9 AA | 0.19% by wt. |
| 10 AA | 0.19% by wt. |
| acrylic acid polymer | 57.12% by wt. |
| fumaric acid | 1.68% by wt. |
| maleic acid | 1.59% by wt. |
| phthalic acid | 0.70% by wt., and |
| | PTZ, MEHQ and conversion products |
| | together were the remainder |
| | to 100% by weight. |

Over the entire treatment, no formation of gas bubbles was observable. At the end of the 3-hour treatment, the liquid present in the four-neck flask with a total amount of 174.5 g had the following composition:

| | |
|---|---|
| monomeric acrylic acid | 5.10% by wt. |
| 2 AA | 4.53% by wt. |
| 3 AA | 3.21% by wt. |
| 4 AA | 2.35% by wt. |
| 5 AA | 1.55% by wt. |
| 6 AA | 0.17% by wt |
| 7 AA | 0.29% by wt. |
| 8 AA | 0.06% by wt. |
| 9 AA | 0.11% by wt. |
| 10 AA | 0.17% by wt. |
| acrylic acid polymer | 76.79% by wt. |
| fumaric acid | 2.06% by wt. |
| phthalic acid | 0.86% by wt., and |
| | PTZ, MEHQ and conversion products |
| | together were the remainder |
| | to 100% by weight. |

In the distillation apparatus, 39.6 g of monomeric acrylic acid had condensed (purity >99% by weight).

### Example 1

Comparative example 1 was repeated with 205.4 g of the same liquid F. During the experiment, however, a gas mixture of 8% by volume of molecular oxygen and 92% by volume of molecular nitrogen was sparged via a long cannula into the lower third of the contents of the four-neck flask (the flask was first heated to 170° C. at atmospheric pressure; then the pressure was reduced to 290 mbar; then the lean air was sparged in at such a rate as to result in a steady-state pressure of 305 mbar).

The total amount of liquid still present in the four-neck flask at the end of the 3-hour treatment was 177.2 g. It had the following composition:

| | |
|---|---|
| monomeric acrylic acid | 3.22% by wt. |
| 2 AA | 4.57% by wt. |
| 3 AA | 2.99% by wt. |
| 4 AA | 1.98% by wt. |
| 5 AA | 1.24% by wt. |
| 6 AA | 0.11% by wt. |
| 7 AA | 0.45% by wt. |
| 8 AA | 0.06% by wt. |
| 9 AA | 0.17% by wt. |
| 10 AA | 0.28% by wt. |
| acrylic acid polymer | 78.89% by wt. |
| fumaric acid | 2.31% by wt. |
| maleic acid | 2.31% by wt. |
| phthalic acid | 0.90% by wt., and |
| | PTZ, MEHQ and conversion products |
| | together were the remainder |
| | to 100% by weight. |

In the distillation apparatus, 28.2 g of monomeric acrylic acid had condensed (purity >99% by weight).

### Example 2

Comparative example 2 was repeated. During the experiment, however, lean air was sparged into the liquid F as in example 1. The initial weight of the liquid F in the four-neck flask was 202.8 g. The composition of the liquid F was, however, as follows:

| | |
|---|---|
| monomeric acrylic acid | 10.31% by wt. |
| 2 AA | 14.30% by wt. |
| 3 AA | 6.21% by wt. |
| 4 AA | 3.11% by wt. |
| 5 AA | 3.50% by wt. |
| 6 AA | 0.20% by wt. |
| 7 AA | 0.39% by wt. |
| 8 AA | 0% by wt. |
| 9 AA | 0.20% by wt. |
| 10 AA | 0.20% by wt. |
| acrylic acid polymer | 56.90% by wt. |
| fumaric acid | 1.68% by wt. |
| maleic acid | 1.68% by wt. |
| phthalic acid | 0.69% by wt., and |
| | PTZ, MEHQ and conversion products |
| | together were the remainder |
| | to 100% by weight. |

The total amount of liquid still present in the four-neck flask at the end of the 3-hour treatment was 149.9 g. It had the following composition:

| | |
|---|---|
| monomeric acrylic acid | 2.20% by wt. |
| 2 AA | 2.94% by wt. |
| 3 AA | 2.07% by wt. |
| 4 AA | 1.40% by wt. |
| 5 AA | 0.73% by wt. |
| 6 AA | 0.07% by wt. |
| 7 AA | 0.20% by wt. |
| 8 AA | 0.07% by wt. |
| 9 AA | 0.13% by wt. |
| 10 AA | 0.20% by wt. |
| acrylic acid polymer | 83.46% by wt. |
| fumaric acid | 2.27% by wt. |
| maleic acid | 2.27% by wt. |
| phthalic acid | 0.93% by wt., and |
| | PTZ, MEHQ and conversion products |
| | together were the remainder |
| | to 100% by weight. |

In the distillation apparatus, 52.9 g of monomeric acrylic acid had condensed (purity >99% by weight).

### Example 3

Comparative example 3 was repeated. During the experiment, however, lean air was sparged into the liquid F as in example 1. The initial weight of the liquid F in the four-neck flask was 196.5 g. The composition of liquid F corresponded to that in example 2. However, the fumaric acid content thereof was 1.78% by weight and the phthalic acid content 0.61% by weight.

The total amount of liquid still present in the four-neck flask at the end of the 3-hour treatment was 121.7 g. It had the following composition:

| | |
|---|---|
| monomeric acrylic acid | 0.33% by wt. |
| 2 AA | 0.16% by wt. |
| 3 AA | 0.08% by wt. |
| 4 AA | 0% by wt. |
| 5 AA | 0% by wt. |
| 6 AA | 0% by wt. |
| 7 AA | 0% by wt. |
| 8 AA | 0% by wt. |
| 9 AA | 0% by wt. |
| 10 AA | 0% by wt. |
| acrylic acid polymer | 91.9% by wt. |
| fumaric acid | 2.88% by wt. |
| maleic acid | 2.22% by wt. |
| phthalic acid | 0.99% by wt., and |
| | PTZ, MEHQ and conversion products |
| | together were the remainder |
| | to 100% by weight. |

In the distillation apparatus 74.8 g of monomeric acrylic acid had condensed (purity >99% by weight).

In all examples 1 to 3, the new formation of acrylic acid polymer associated with the thermal treatment is significantly lower than the case of the thermal treatment carried out at the corresponding temperature in the comparative example.

Instead of lean air, residual gas removed from the condensation column could also have been used in examples 1 to 3.

### Example 4

Comparative example 1 was repeated with 207.0 g of liquid F from example 2. During the experiment, however, beginning with the adjustment of the pressure to 290 mbar, with the aid of a Prominent pump, an acid water stream of strength 10 g/3 h was metered into the lower third of the contents of the four-neck flask.

The acid water was condensed into the upper part of the condensation column.

| | | |
|---|---|---|
| It comprised: | water | 81.9% by wt. |
| | acrylic acid | 9.4% by wt. |
| | acetic acid | 4.1% by wt. |
| | formaldehyde | 3.8% by wt. |
| | formic acid | 0.7% by wt. |
| | propionic acid | 0.01% by wt., and |
| | MEHQ | 0.0001% by wt. |

The total amount of liquid still present in the four-neck flask at the end of the 3-hour treatment was 179.1 g. It had the following composition:

| | |
|---|---|
| monomeric acrylic acid | 7.93% by wt. |
| 2 AA | 7.98% by wt. |
| 3 AA | 5.08% by wt. |
| 4 AA | 2.07% by wt. |
| 5 AA | 1.56% by wt. |
| 6 AA | 0.17% by wt. |
| 7 AA | 0.06% by wt. |
| 8 AA | 0.11% by wt. |
| 9 AA | 0.17% by wt. |
| 10 AA | 0.22% by wt. |
| acrylic acid polymer | 69.12% by wt. |
| fumaric acid | 1.90% by wt. |
| maleic acid | 1.90% by wt. |
| phthalic acid | 0.78% by wt., and |
| | water, PTZ, MEHQ and conversion |
| | products together were the remainder |
| | to 100% by weight. |

In the distillation apparatus, a condensate was condensed which comprised 21.5 g of monomeric acrylic acid and 9.6 g of water.

### Example 5

Comparative example 2 was repeated. In addition, the acid water stream from example 4 was metered in as in example 4: the amount of liquid F initially charged was 201.1 g.

The composition of liquid F was:

| | |
|---|---|
| monomeric acrylic acid | 12.33% by wt. |
| 2 AA | 15.07% by wt. |
| 3 AA | 6.76% by wt. |
| 4 AA | 3.38% by wt. |
| 5 AA | 3.58% by wt. |
| 6 AA | 0.20% by wt. |
| 7 AA | 0.35% by wt. |
| 8 AA | 0.15% by wt. |
| 9 AA | 0.15% by wt. |
| 10 AA | 0.10% by wt. |
| acrylic acid polymer | 54.25% by wt. |
| fumaric acid | 1.39% by wt. |
| maleic acid | 1.39% by wt. |
| phthalic acid | 0.50% by wt., and |
| | water, PTZ, MEHQ and conversion |
| | products together were the remainder |
| | to 100% by weight. |

The total amount of liquid still present in the four-neck flask at the end of the 3-hour treatment was 137.4 g. It had the following composition:

| | |
|---|---|
| monomeric acrylic acid | 4.29% by wt. |
| 2 AA | 4.15% by wt. |
| 3 AA | 2.47% by wt. |
| 4 AA | 1.82% by wt. |
| 5 AA | 0.58% by wt. |
| 6 AA | 0.15% by wt. |
| 7 AA | 0.07% by wt. |
| 8 AA | 0.07% by wt. |
| 9 AA | 0.07% by wt. |
| 10 AA | 0.07% by wt. |
| acrylic acid polymer | 80.57% by wt. |
| fumaric acid | 2.04% by wt. |
| maleic acid | 2.04% by wt. |
| phthalic acid | 0.73% by wt., and |
| | water, PTZ, MEHQ and conversion |
| | products together were the remainder |
| | to 100% by weight. |

In the distillation apparatus a condensate was condensed which comprises 63.7 g of monomeric acrylic acid and 9.8 g water.

Both in example 4 and in example 5, the new formation of acrylic acid polymer associated with the thermal treatment is significantly lower than in the case of the thermal treatment carried out at the corresponding temperature in the comparative example.

U.S. Provisional Patent Application No. 61/048,334, filed Apr. 28, 2008, is incorporated into the present patent application by literature reference. With regard to the abovementioned teachings, numerous changes and deviations from the present invention are possible. It can therefore be assumed that the invention, within the scope of the appended claims, can be performed differently from the way described specifically herein.

1. A process for transferring heat to a liquid F comprising dissolved monomeric acrylic acid, Michael acrylic acid oligomers and acrylic acid polymer with the aid of an indirect heat exchanger having at least one primary space and at least one secondary space separated from the at least one primary space by a material dividing wall D, in which the liquid F flows through the at least one secondary space, while the at least one primary space is simultaneously flowed through by a fluid heat carrier W, where the liquid F flows into the at least one secondary space with a temperature T^{F}≧130° C. and the fluid heat carrier W into the at least one primary space with a temperature T^{W}>T^{F}, wherein
a) the liquid F on entry into the at least one secondary space comprises
5 to 50% by weight of Michael acrylic acid oligomers,
at least 40% by weight of acrylic acid polymer,
up to 25% by weight of monomeric acrylic acid,
up to 2% by weight of polymerization inhibitor, and
up to 15% by weight of other compounds,
and
b) in the liquid F flowing through the at least one secondary space, during the flow through the at least one secondary space, the formation of gas bubbles and/or of thin layers of the liquid F adjoining a gas phase is brought about.

2. The process according to claim 1, wherein the liquid F on entry into the at least one secondary space comprises
5 to 50% by weight of Michael acrylic acid oligomers,
40 to 80% by weight of acrylic acid polymer,
5 to 20% by weight of monomeric acrylic acid,
0.1 to 2% by weight of polymerization inhibitor, and
1 to 15% by weight of other compounds.

3. The process according to claim 1, wherein the liquid F on entry into the at least one secondary space comprises
10 to 40% by weight of Michael acrylic acid oligomers,
50 to 70% by weight of acrylic acid polymer,
5 to 15% by weight of monomeric acrylic acid,
0.1 to 1% by weight of polymerization inhibitor, and
1 to 15% by weight of other compounds.

4. The process according to claim 1, wherein the liquid F comprises
15 to 35% by weight of Michael acrylic acid oligomers,
50 to 70% by weight of acrylic acid polymer,
5 to 15% by weight of monomeric acrylic acid,
0.1 to 1% by weight of polymerization inhibitor, and
1 to 15% by weight of other compounds.

5. The process according to any one of claims 1 to 4, wherein the total amount of the Michael acrylic acid oligomers present in the liquid F on entry into the at least one secondary space consists of Michael acrylic acid dimers to an extent of from 40 to 60% by weight.

6. The process according to any one of claims 1 to 5, wherein the total amount of the Michael acrylic acid oligomers present in the liquid F on entry into the at least one secondary space consists of Michael acrylic acid trimers to an extent of from 15 to 30% by weight.

7. The process according to any one of claims 1 to 6, wherein the number-average molecular weight of the acrylic acid polymer present in the liquid F on entry into the at least one secondary space is from 500 to 10⁶.

8. The process according to any one of claims 1 to 6, wherein the number-average molecular weight of the acrylic acid polymer present in the liquid F on entry into the at least one secondary space is from 750 to 750 000.

9. The process according to any one of claims 1 to 6, wherein the number-average molecular weight of the acrylic acid polymer present in the liquid F on entry into the at least one secondary space is from 1000 to 100 000.

10. The process according to any one of claims 1 to 9, wherein the total amount of compounds from the group consisting of fumaric acid, maleic acid, phthalic acid and the anhydrides of these carboxylic acids present in the liquid F on entry into the at least one secondary space is ≦10% by weight.

11. The process according to any one of claims 1 to 10, wherein the liquid F on entry into the at least one secondary space comprises at least one added substance whose boiling point at standard pressure is below that of acrylic acid.

12. The process according to claim 11, wherein the liquid F comprises, as the added substance present, water and/or an aqueous solution.

13. The process according to claim 12, wherein the liquid F comprises, as the added substance present, acid water.

14. The process according to any one of claims 1 to 13, wherein at least one substance whose boiling point at 1 atm is ≦−40° C. has been metered into the liquid F before it enters the at least one secondary space.

15. The process according to any one of claims 1 to 13, wherein residual gas has been metered into the liquid F before it enters the at least one secondary space.

16. The process according to any one of claims 1 to 15, wherein 130° C.≦T^{F}≦250° C.

17. The process according to any one of claims 1 to 15, wherein 150° C.≦T^{F}≦225° C.

18. The process according to any one of claims 1 to 15, wherein 160° C.≦T^{F}≦200° C.

19. The process according to any one of claims 1 to 18, wherein the difference ΔT^{A,F} between the temperature T^{F} and the temperature T^{A} with which the heated liquid leaves the at least one secondary space is from 0.1 to 70° C.

20. The process according to any one of claims 1 to 18, wherein the difference ΔT^{A,F} between the temperature T^{F} and the temperature TA with which the heated liquid leaves the at least one secondary space is from 5 to 50° C.

21. The process according to any one of claims 1 to 20, wherein T^{W}-T^{F} is from 1 to 150° C.

22. The process according to any one of claims 1 to 20, wherein T^{W}-T^{F} is from 5 to 100° C.

23. The process according to any one of claims 1 to 20, wherein T^{W}-T^{F} is from 20 to 60° C.

24. The process according to any one of claims 1 to 23, wherein, based on the total volume of the at least one secondary space flowed through by the liquid F, from 0.1 to 25% by volume is accounted for by gas bubbles present therein and from 99.9 to 75% by volume by liquid phase present therein.

25. The process according to any one of claims 1 to 23, wherein, based on the total volume of the at least one secondary space flowed through by the liquid F, from 0.5 to 15% by volume is accounted for by gas bubbles present therein and from 99.5 to 85% by volume by liquid phase present therein.

26. The process according to any one of claims 1 to 25, wherein the liquid F is forcibly conveyed through the at least one secondary space.

27. The process according to any one of claims 1 to 26, wherein the indirect heat exchanger is a tube bundle heat transferrer.

28. The process according to any one of claims 1 to 27, wherein the indirect heat exchanger is a multiflow tube bundle heat transferrer.

29. The process according to any one of claims 1 to 26, wherein the indirect heat exchanger is a plate heat transferrer.

30. The process according to any one of claims 1 to 26, wherein the indirect heat exchanger is a thin film heat transferrer.

31. The process according to any one of claims 1 to 26, wherein the indirect heat exchanger is a falling film heat transferrer.

32. The process according to any one of claims 1 to 26, wherein the indirect heat exchanger is a helical tube evaporator.

33. The process according to any one of claims 1 to 32, wherein the liquid F on entry into the at least one secondary space comprises at least one added active substance which lowers the interface tension for the formation of a gas bubble in the liquid F.
